(19) United States
(12) Patent Application Publication
(10) Pub. No.: US 2025/0327013 A1
(43) Pub. Date: Oct. 23, 2025

(54) System and Method for Quantifying Oxygen Production and Consumption of Suspended Particles and Organisms

(71) Applicant: Woods Hole Oceanographic Institution, Woods Hole, MA (US)

(72) Inventors: Clarissa KARTHAEUSER, Falmouth, MA (US); Paul Dominic FUCILE, Waquoit, MA (US)

(21) Appl. No.: 19/173,914

(22) Filed: Apr. 9, 2025

Related U.S. Application Data
(60) Provisional application No. 63/637,077, filed on Apr. 22, 2024.

(51) Int. Cl. C12M 1/34; C12M 1/24; C12M 3/00; C12M 3/04

(52) U.S. Cl. CPC C12M 41/34; C12M 23/08; C12M 23/42; C12M 23/48; C12M 27/10

(57) ABSTRACT

A system and method utilizing a rotating incubator enabling automated measurements of one or more selected parameters such as oxygen in small volumes while preserving natural suspension states of planktonic items in liquid samples. This allows highly sensitive rate measurements of oxygen production or utilization and subsequent characterization of single particles, heterogeneous samples such as microplastics, microbial cultures or sediment slurry, or small planktonic organisms, such as copepods, jellyfish, or protists.

### CROSS-REFERENCE TO RELATED APPLICATION

This application claims priority to U.S. Provisional Application No. 63/637,077 filed on 22 Apr. 2025. The entire contents of the above-mentioned application are incorporated herein by reference as if set forth herein in entirety.

### FIELD OF THE INVENTION

This invention relates to systems and methods of determining oxygen concentrations in samples over time.

### BACKGROUND OF THE INVENTION

Oxygen respiration is the main metabolic process of many organisms and an essential factor in the breakdown of organic matter and release of carbon dioxide (CO₂). Particles and sub-millimeter scale zooplankton represent small respiration hotspots that affect carbon flux and organic matter decomposition throughout the water column. At present, there is a lack of understanding of the factors that drive metabolic rates by these hotspots. As a result, carbon-flux estimates have large uncertainties, carbon and energetic budgets are not balanced, and future metabolic-related changes are difficult to predict.

Diverse factors drive metabolic rates, including individual properties of the particle or organism, such as its (i) elemental and microbial community composition and packaging in the case of particles and (ii) life stage, health, and sex in the case of organisms. Further, external factors such as temperature and partial pressures of oxygen and carbon dioxide play important roles. Therefore, controlled rate measurements of oxygen respiration on the single-particle and single-organism scale are key towards a mechanistic understanding of marine carbon fluxes.

Oxygen respiration rates in marine samples are most commonly measured using sensors that detect oxygen concentrations over time. These include highly sensitive optical systems detecting oxygen concentrations in the nanomolar range, and micro-electrode sensors to determine small-scale gradients. See, e.g., U.S. Pat. Nos. 9,188,512 and 10,486,991 by Van Mooy et al.

Optode sensors detect oxygen concentrations by sending a light impulse into a sensor material that quenches the light based on the surrounding oxygen concentrations without consuming oxygen. The accuracy of optode-based oxygen detection during in situ seawater incubations has greatly been improved through on-the-spot temperature corrections, but this new technology has not been implemented yet in most studies. Using optode sensors, previous studies have contributed substantially to an understanding of respiration in model particles, small organisms and pooled biomass. Examples for particles include the respirometry setup by Stief et al., which allows for oxygen drawdown measurements in four rotating vials under a range of pressure conditions. See, e.g., Stief et al., Respiration by “marine snow” at high hydrostatic pressure: Insights from continuous oxygen measurements in a rotating pressure tank, Limnol. Oceanogr. 2021 July; 66(7):2797-809 and Stief et al., Hydrostatic pressure induces transformations in the organic matter and microbial community composition of marine snow particles, Communications Earth & Environment. 2023 Oct. 14;4(1):1-4.

Another recent approach is the RESPIRE sediment trap, which has been engineered to intercept and incubate pooled particles at depth, allowing for natural temperature and pressure settings. See Boyd et al., RESPIRE: An in-situ particle interceptor to conduct particle remineralization and microbial dynamics studies in the oceans' Twilight Zone, Limnol. Oceanogr. Methods, 2015 September;13(9):494-508. However, particles in the RESPIRE trap are incubated as a pooled sample rather than individually, while resting rather than sinking. Options to incubate individual particles and organisms at high replication and sensitivity while providing close to natural conditions are currently missing.

Sinking and floating are important factors when studying the activity of particles or organisms that reside in the water column. Metabolic processes, such as respiration in particles, are supplied with substrates, while released products are removed through the surrounding water flow and pore water flow. High particle-associated respiration and low oxygen supply can lead to the formation of anoxic micro-niches. Further, sinking is an important factor that drives interaction with microbial communities and formation of consortia on particles. For metazoans, keeping them close to their natural conditions is essential to determine accurate oxygen respiration rates.

### SUMMARY OF THE INVENTION

An object of the present invention is to enhance the accuracy of oxygen respiration measurements for liquid samples containing particulates and/or living planktonic organisms.

The present invention relates to a rotating laboratory incubator such as the RotoBOD™ incubator, configured to rotate and determine oxygen concentrations over time such as Biological Oxygen Demand (BOD). The present invention uniquely enables highly sensitive automated oxygen measurements in small volumes while keeping the samples as close as possible to their natural state of moving actively or passively through water. Automated sensor positioning and on-the-spot temperature detection allow precise rate measurements of oxygen utilization and subsequent individual characterization of a plurality of concurrent samples. The diverse types of samples that can be incubated in the RotoBOD™ include single particles and small planktonic organisms such as copepods, jellyfish, coccolithophores, planktonic foraminifera and others, for which such information is currently limited.

This invention features a system to incubate and monitor liquid samples over a selected time period, including at least one rotatable cassette having an axis of cassette rotation and configured to hold a plurality of sample vials which are held radially outwardly from the axis of cassette rotation along a plurality of spaced radii relative to the axis of cassette rotation. Each vial has a sensing location and a longitudinal axis alignable by the cassette with one of the spaced radii to position its sensing location proximal to the axis of cassette rotation. The system further includes a sensor configured to measure at least one parameter and having a sensor head, and a first motor configured to rotate the cassette about its axis of cassette rotation. A controller actuates the motor to periodically align the sensing location of each vial with the sensor head to measure at least one parameter of that vial.

In some embodiments, each vial has at least one optode spot at its sensing location. In certain embodiments, the system includes at least two cassettes, and the sensor is mounted on a sensor track to enable shuttling between the cassettes. The sensor track may include at least one guide rail and an advancement mechanism which includes a second motor configured to shuttle the sensor between the cassettes. In one embodiment, the cassettes are rotated together by the first motor in fixed alignment with each other.

In a number of embodiments, the sensor measures at least oxygen concentration of the liquid sample within each vial. In some embodiments, the temperature of the liquid sample within each vial is determined when the oxygen concentration is measured. In certain embodiments, the system is installed in a custom incubator chamber with temperature control by a mechanism such as convection in an air bath. In other embodiments, temperature control is provided independently for one or more vials. In certain embodiments, at least one cassette is configured with a releasable vial locking mechanism to enable selective individual removal and replacement of at least one of the vials from the cassette assembly.

This invention also features a method for incubating and monitoring liquid samples over a selected time period, including selecting at least one rotatable cassette having an axis of cassette rotation and configured to hold a number of sample vials, with the vials held radially outwardly from the axis of cassette rotation along a plurality of spaced radii relative to the axis of cassette rotation, and each vial having a sensing location and having a longitudinal axis alignable by the cassette with one of the spaced radii to position its sensing location proximal to the axis of cassette rotation. A liquid sample is placed in each of a plurality of sample vials. The cassette is rotated at a selected rotation speed at selected intervals to achieve a selected sampling period for each vial. At least one parameter within each vial is measured periodically utilizing a sensor head by periodically aligning the sensing location of each vial with the sensor head to measure the at least one parameter of that vial.

In some embodiments, the rotation speed is selected to be sufficiently high to prevent planktonic items in the liquid sample within each vial from accumulating on any surface of that vial.

### BRIEF DESCRIPTION OF THE DRAWINGS

To enable a better understanding of the present invention, and to show how the same may be carried into effect, certain embodiments of the invention are explained in more detail with reference to the drawings, by way of example only, in which:

FIG. 1A is a schematic perspective view of a RotoBOD™ rotating incubator according to the present invention including a sensor unit with sensor head and a rotatable cassette assembly;

FIG. 1B is a schematic perspective view of the cassette assembly of FIG. 1A;

FIG. 1C is a schematic perspective view of one of the cassettes in the assembly of FIGS. 1A-1B;

FIG. 1D is a schematic enlarged perspective view of the sensor unit of FIG. 1A with a sample vial to be read;

FIG. 1E is a schematic enlarged perspective view of an alternative vial and sensor unit configuration;

FIG. 1F is a schematic diagram of a number of the electrical and mechanical components of FIG. 1A;

FIG. 1G is a schematic diagram similar to FIG. 1F of a system utilizing the sensor unit of FIG. 1E;

FIG. 2A is a schematic side view of four vial positions depicting an optimal rotation path of a particle during incubation;

FIG. 2B is a chart showing oxygen drawdown measured in an unfiltered seawater sample over an incubation period of 70 hours;

FIGS. 3A and 3B are charts depicting the effect of rotation on respiration rates in small copepods and marine snow particles;

FIG. 4 is a chart depicting measured respiration rates in seawater from samples collected at various depths;

FIG. 5A is a chart depicting oxygen respiration of individual Calanopia americana copepods correlated with the animal's biovolume;

FIG. 5B is a chart showing the ratio between measured respiration and size-based predicted respiration in female and male individuals, and juvenile copepods or animals for which the sex could not be determined; and

FIG. 6 is a schematic perspective view of a bottle holder with releasable locking mechanism utilized according to some embodiments of the present invention.

### DETAILED DESCRIPTION OF THE PRESENTLY PREFERRED EMBODIMENTS

This invention may be accomplished by a system referred to as RotoBOD™ rotating incubator enabling automated oxygen measurements in small volumes while keeping the samples in their natural state of suspension. In some constructions, the RotoBOD™ is configured to rotate and determine Biological Oxygen Demand (BOD) for individual containers such as sample vials or bottles. Besides measuring respiration at high accuracy, it can be used to determine photosynthetic oxygen production, and enable anaerobic incubations under controlled conditions.

The term “planktonic items” as utilized herein includes particulates and/or organisms that are generally suspended or otherwise drift in water but are unable to actively propel themselves against currents in their native environments.

Systems and methods according to the present invention are non-destructive and achieve highly increased survival rates of incubated planktonic items. The present invention enables parallel and subsequent analyses such as stable isotope additions and molecular biology, and diverse other measurements to characterize incubated particles and/or small planktonic organisms. Further, the RotoBOD™ incubator may be equipped with additional sensors, including light detection, carbon dioxide and pH sensor spots. The flexible design and on-the-spot temperature detection allow for variations in bottle size and incubator setups such as placing the RotoBOD™ incubator in a temperature gradient.

The present robotic incubator is capable of measuring oxygen concentrations while simulating natural conditions for planktonic items utilizing rotation. Various options arise for the use of the RotoBOD™, such as detecting respiration on newly introduced materials like microplastics, detecting photosynthetic activity by incubating with light, combining oxygen respiration with measurements of other processes by adding stable isotopes such as 15N-and 13C-compounds, obtaining high-resolution respiration rates over the diel cycle of small zooplankton, studying particles from hydrothermal vents and their role for the deep ocean, quantifying the breakdown of relevant contributors to carbon export such as macroalgae, and many others with minimal efforts due to the high level of automation.

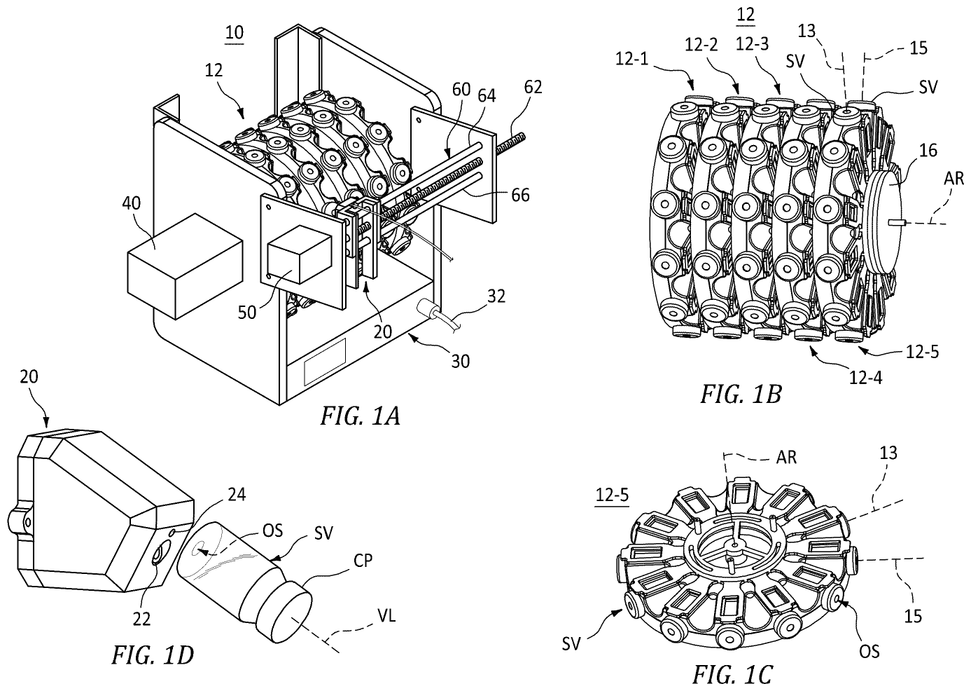

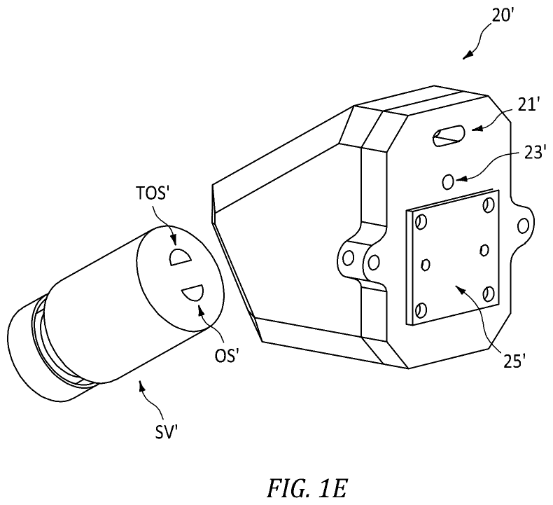

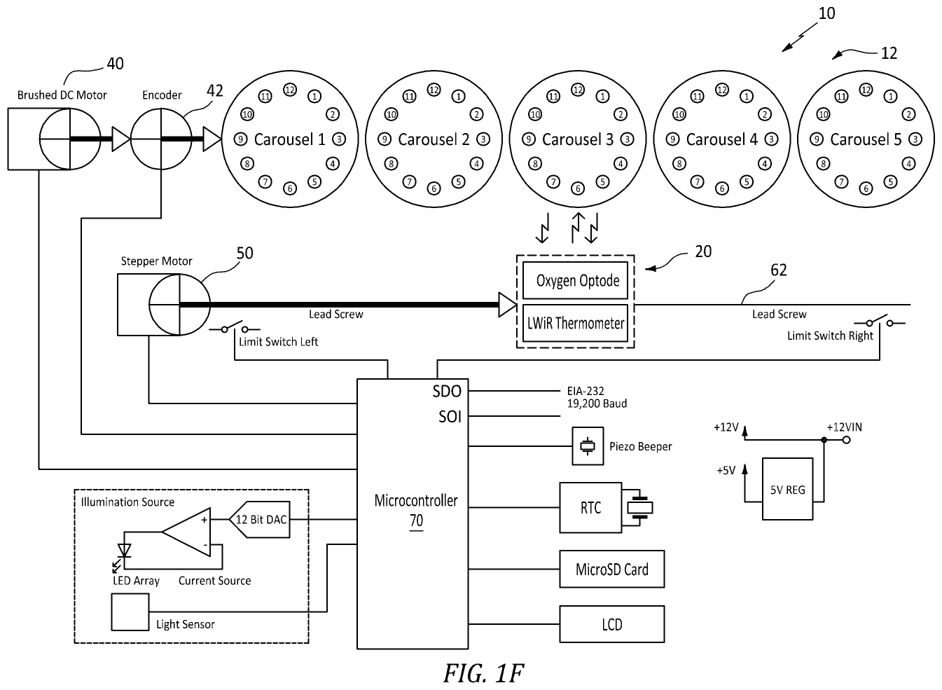

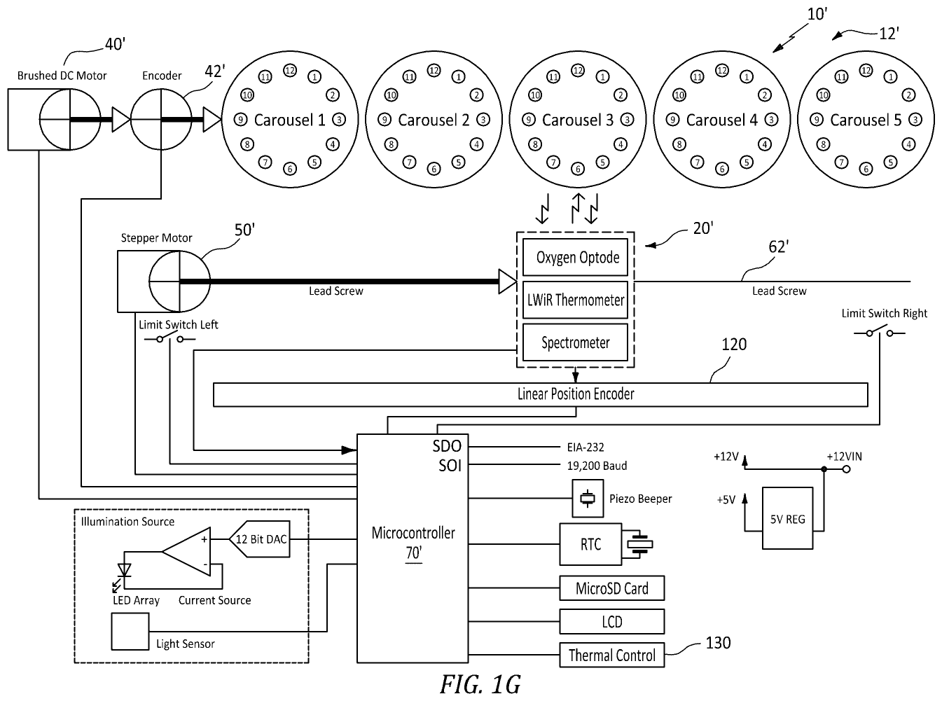

FIGS. 1A-1D depict a schematic overview of one construction of a RotoBOD™ system 10 demonstrating precise oxygen sensing technology through automated positioning of a sensor head 22 within a sensor unit 20 and on-the-spot temperature correction while utilizing rotation to meet the requirements of incubating small samples containing planktonic items. Five removable cassettes 12-1 through 12-5 of a cassette assembly 12 having a central hub 16 hold a total of sixty gas-tight 10 mL incubation vials SV, each vial equipped with an optode spot OS opposite a cap CP as shown in FIG. 1D.

As indicated for rotatable cassette 12-5 in FIGS. 1B-1C, each cassette shares an axis of cassette rotation AR with cassette assembly 12 and is configured to hold twelve sample vials SV in this construction, with the vials SV held radially outwardly from the axis of cassette rotation along a plurality of spaced radii such as radii 13, 15 relative to the axis of cassette rotation AR. Interlocking features such as detents and recesses (e.g., pins and matching holes) are provided on opposing sides of each cassette in certain constructions. Each vial has a sensing location such as optode spot OS and has a vial longitudinal axis VL, FIG. 1D, alignable by the cassette with one of the spaced radii 13, 15, for example, to position its sensing location proximal to the axis of cassette rotation AR.

Sensor unit 20, also referred to herein as a detector assembly, holds an optode fiber and communicates with a microcontroller on a circuit board inside an aluminum casing 30 having one or more access cables 32, FIG. 1A, as described in more detail below in relation to FIG. 1F. Port 24, FIG. 1D, includes a distal end of a light fiber as described below. High torque brushed gear motor 40, FIG. 1A, provides controlled rotation to keep samples afloat and position each bottle in front of the sensor head 22. Linear stepper motor 50 controls horizontal sensor head movement between the cassettes along lead screw 62 as supported by upper and lower guide rails 64, 66.

In one construction, the RotoBOD™ is equipped with a high-torque brushed GEA motor 40 to rotate 60 gas-tight incubation vials (10 mL serum vials, closed with WHEATON® straight-plug Chlorobutyl/Isoprene, PTFE/Butyl stoppers and sealed with aluminum crimp rings to form cap CP), held by five 3D-printed 12-bottle cassettes (printed with acrylonitrile styrene acrylate fiber on a Stratus Fortus 450 mc), around a horizontal axis of rotation AR at adjustable speeds to keep particles and small organisms in suspension. Each vial is equipped with an oxygen sensing spot OS (Presens® SP-PSt3-NAU, 5 mm in diameter) that is designed to quench light based on oxygen concentrations.

In one construction, the RotoBOD™ utilizes a Presens Electro-Optical Module (Presens® EOM-O2-FOM) for sensor unit 20 with a light fiber 24 (Presens® POF-L2.5-1SMA) as a sensor head that both emits a green flash through port 24 and directs the reflected and quenched light impulse back to the sensor. When optical sampling is selected, the RotoBOD decreases its rotation velocity whenever the sensor head approaches a vial sensor spot and places the light fiber cable on the center of each spot, based on the amplitude of the detected signal, which is highest at the center of each spot. This enables the RotoBOD™ to avoid variations in manual measurement techniques that previously introduced variability to oxygen-concentration measurements.

Further, horizontal movement of the oxygen sensor is realized through a second motor 50 (NEMA 17 External Linear Actuator Stepper) with a lead screw 62, resulting in precisely replicable placement of the sensor on the spots. The full automation further enables the instrument to be installed in a custom incubator chamber with temperature control by convection in an air bath, which, together with the small vial volume, results in a very high sensitivity. Temperature is detected proximate to the spot through long-wave infrared sensing, which enables accurate local temperature corrections for oxygen solubility. In other constructions, temperature control is provided independently for one or more vials.

An alternative detector assembly 20′, FIG. 1E, has an optode fiber entry port 21′, an infra-read thermometer wiring entry port 23′, and a spectrometer 25′ to accommodate sample bottle SV′ having dual sensing spots: a standard oxygen sensor spot OS′ and a trace oxygen sensor spot TOS′. This enables measurement of lower and standard oxygen levels with the same sample bottle and the ability to measure luminescence or incubator light levels and spectra.

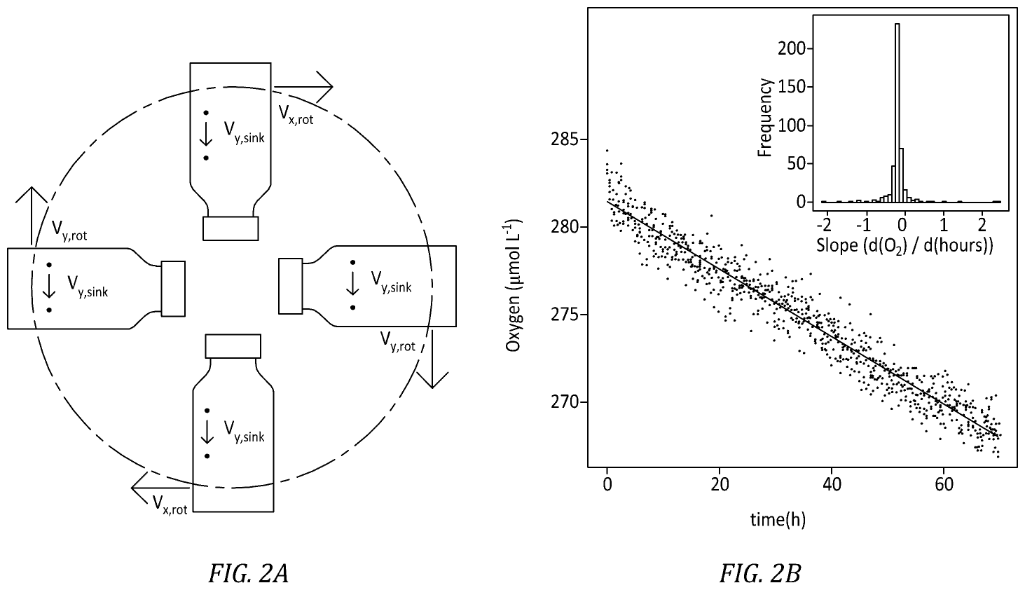

A number of electrical and mechanical components of RotoBOD™ system 10 are schematically depicted in FIG. 1F depicting interactions among the components. Sensor unit 20 with an Oxygen Optode and a LWIR Thermometer is shown optically reading a vial in cassette 12-3 after Brushed DC Motor 40 has stopped rotation of cassette assembly 12 as commanded by Microcontroller 70 and monitored by rotary Encoder 42. Operation of system 10 is described in more detail below in relation to FIGS. 2A-2B.

FIG. 1G is a schematic diagram similar to FIG. 1F of a system utilizing the sensor unit 20′ of FIG. 1E. The stepper motor 50′ moves the sensor unit 20′ horizontally from cassette to cassette. The linear position encoder 120 provides feedback to the controller 70′ to determine which cassette is being sampled. In some constructions position feedback is performed by counting steps but in other, more precise constructions, position encoders are utilized to precisely locate the Optode fiber over the sensor spot on the selected sample bottle. The Brushed DC motor 40′ uses rotary encoder 42′ to similarly inform the system 10′ what bottle is being observed. Both together provide precision XY knowledge.

System 10′ also enables temperature control of the sample bottles utilizing Thermal Control 130, FIG. 1G. In one construction, one or more solid state Peltier devices are utilized to adjust temperature, such as described by P. Fucile in U.S. Pat. No. 12,055,641, for example.

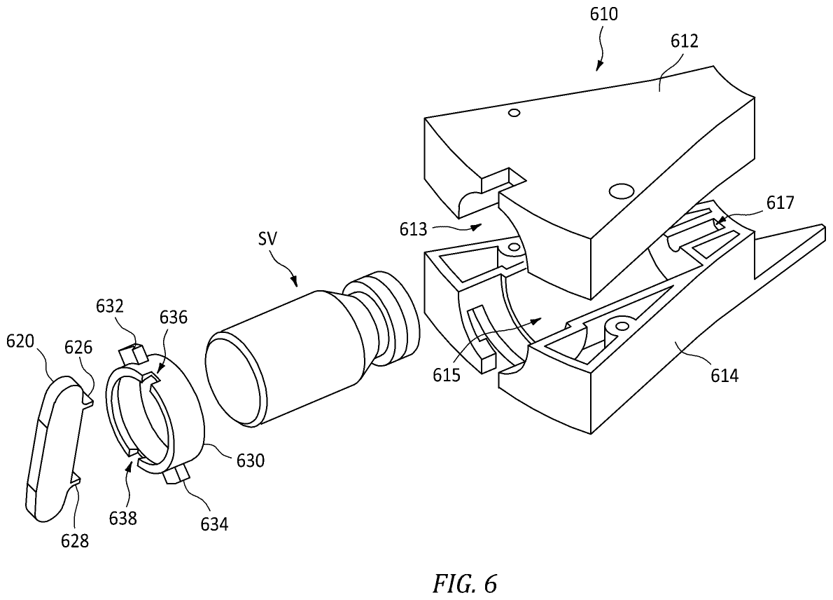

Sample bottle holder 610, FIG. 6, also referred to as a “wedge”, is utilized in cassettes in some constructions to accommodate different sizes of bottles to be incubated and monitored according to the present invention. Bottle holder 610, when carried by a cassette, also enables users to add and remove individual bottles without disassembling the cassette assembly or otherwise interrupting incubation of the remaining bottles. Upper section 612 defines a cavity 613 and lower section 614 defines a cavity 615 to accept bottle SV. A locking mechanism including a locking key 620 and a retaining collar 630 assist positioning and removable retention of bottle SV within sections 612 and 614. In the illustrated construction, locking key 620 has detents 626 and 628 which interlock with recesses 636 and 638 of retaining collar 630 after its bayonet tabs 632 and 634 are engaged with matching slots defined by sections 612 and 614, respectively.

In some constructions, one or both of the sections of the wedge 610 define a retention feature such as a channel 617 in section 614 to hold a biasing element such as a coil spring to provide retention resistance against the cap of a bottle SV. Securing the sections 612 and 614 together with fasteners or other interlocking features serves to clamp the coil spring to anchor it in a selected position during use of the wedge.

FIG. 2A schematically depicts an optimal rotation path of a particle in a vial during incubation among four opposed positions at different times in a full rotation of a cassette holding the vial. The particle sinks in vertical (y) direction and is repeatedly lifted back towards the top of the vial through the rotation. Ideally, horizontal movement (x) balances to zero. As a result, the particle is continuously lifted in vertical direction and, due to its gravitational sinking, performs a small rotation within the bottle, the path of which is illustrated among the four opposed positions at the different time periods. Further sinking and suspension considerations relating to size and buoyancy of sampled planktonic items and selected rotation velocity are presented below.

FIG. 2B illustrates an example of oxygen drawdown measured in an unfiltered seawater sample over an incubation period of 70 hours. Oxygen concentrations were measured every 20 minutes at 5-fold replication of the light pulse. The resulting oxygen drawdown rate was estimated using a Monte-Carlo-simulation. Slopes between randomly sampled individual data points resulted in a normal distribution, of which the mean represents the linear slope of the oxygen drawdown, and the standard error represents the variability of the measurement, depicted as error bars on oxygen respiration rates.

The use and features of the present system and the importance of keeping samples of planktonic items in suspension were demonstrated using seawater from the upper 1,000 m of the mesopelagic Atlantic. The samples included Calanoid copepods of the species Calanopia americana, sampled off Bermuda, deep-sea copepods of the species Stygiopontius hispidulus, and detritus particles from a shallow marine bay. The Bermuda Atlantic Time-series Study site (BATS) provides a well-characterized location in the subtropical North Atlantic (32° 10′N, 64° 30′ W) that is sampled regularly at a high temporal and spatial resolution, which makes it an ideal location for testing this new method on seawater samples and embedding it in the context of previous approaches. Additional sampling details are provided below.

Particles in the ocean or other liquid body sink or float, depending on their buoyancy. The sinking velocity of a particle is not only driven by its size but various other properties such as compactness and ballasting. Typical sinking velocities of marine snow particles are on average around 100 m/day and range between close to zero (suspended particle) and up to 1000 m day−1. By comparison, other planktonic items such as many microplastics have a positive buoyancy and will accumulate on an upper surface unless rotated.

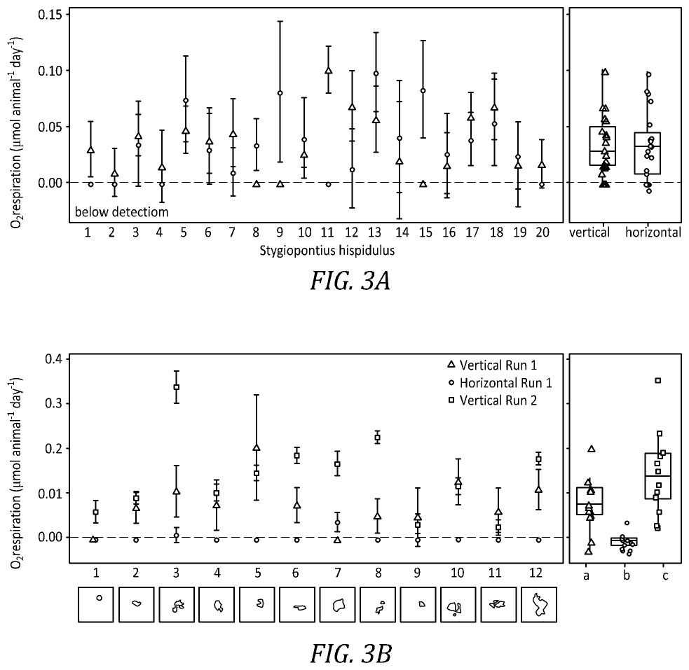

If a sinking particle is kept in the RotoBOD™ system, it performs a small rotation within the bottle, depending on the bottle's rotation velocity and the particle's sinking velocity (FIG. 2A). The slower the RotoBOD™ rotates and the faster the particle sinks, the larger is the radius of its circular path within the bottle. While the rotation velocity of the RotoBOD™ and the number of measurements in between constant rotation can be adjusted, incubating up to 60 individual particles at a time utilizing a single drive motor comes with the practical limitation that these parameters cannot be adjusted for the individual particle. The rotation velocity of the RotoBOD™ cassettes is limited by the need to stop the rotation accurately whenever the sensor detects an optode spot. The currently fastest setting advances the RotoBOD™ sensor from one bottle to the next within 14 seconds, where the sensor spends 7 seconds measuring. As a result, the average particle (100 m/s sinking velocity) will remain suspended, and even a particle with a very high sinking velocity that will not remain suspended throughout the incubation will move along the wall of the vial since their ideal rotation path is too large for the vial, pausing during the 7 seconds of measurement. This will enable an exchange of metabolites with the surrounding water and help prevent the particle from resting on the bottom of the vial, accumulating with additional particles or other planktonic items on that surface, and forming an anoxic micro-layer. The tests suggest that this results in increased respiration rates compared to resting particles (FIGS. 3A-3B).

In general, higher sampling resolution is usually preferable. Rotation velocity, also referred to herein as rotation speed, is limited on the lower end by the need to keep planktonic items in suspension within each sample vial by balancing sinking speed as illustrated in FIG. 2A. At the upper end, the fastest rotation is limited by sensor sampling characteristics including stopping accurately in the optimal reading position for a sufficient period of time to obtain a reading.

To operate one construction of the RotoBOD™ system, a user selects a sampling period such as reading each vial once every 20 minutes. The number of vials to be sampled is determined as well as the sampling time per vial. The cassettes are rotated to match the sensor with each selected vial to achieve the sampling period. The sensor is moved among cassettes as needed to align with each selected vial. In some constructions, the user chooses the number of samples and measures at the highest resolution possible with that number and the instrument's maximum speed.

Organisms up to approximately one cm in diameter have been incubated in the RotoBOD™ system of FIGS. 1A-1D, but they drew down oxygen very fast, so the minimum and maximum respiration rates within each sample vial are more of a limitation than organism size itself. Particles<150 μm often do not respire enough for the signal to be detected, while too large/active organisms may respire so fast that the oxygen is consumed within 1-2 hours and the rate has too few data points and therefore a high error/low statistical significance. Microbial cultures contain lots of tiny cells but may have a high respiration.

FIGS. 3A-3B depict the effect of rotation on respiration rates in small copepods and marine snow particles. As illustrated in FIG. 3A, twenty individual benthic copepods (Stygiopontius hispidulus, with individuals designated by number 1-20 along the x-axis) did not show significantly different respiration rates under horizontal cassette rotation (no perceived movement when the RotoBOD™ system was reoriented ninety degrees to have its axis of rotation vertical) compared to vertical cassette rotation (simulated sinking, Student's t-test: p=00.95, n=20). A median blank rate of −0.017 μmol O2 animal−1 day−1 was detected in four 0.2-μm-filtered seawater samples and subtracted from the measured rates. Rates that were below the blank rate were considered below detection and are shown as zero rates below the dashed line.

Respiration rates in individual marine snow particles under horizontal and vertical rotation are illustrated in FIG. 3B. Respiration was measured while keeping the particles in a sinking motion through rotation to simulate “vertical” (vertical run 1), followed by an incubation rotated by 90 degrees, resulting in the particles resting on the side of the vial as “horizontal”, and a second vertical rotation, vertical run 2. All data are shown in the left panel, while data are grouped in plots “a”-“c” according to horizontal and vertical rotation runs in the right panel. A blank of 0.2−μm-filtered seawater was made and incubated as a blank; a resulting median background rate of 0.07 μmol particle-1 day-1 was subtracted from the particle rates. As a result, rates in the section of the plots below the dashed line are below detection. Particles were collected from a shallow marine bay during tidal outflow at Woods Hole, MA. Schematic images of each of the twelve particles are shown below the rates along the x-axis.

In the case of zooplankton such as jellyfish or copepods, the rotation may help simulate movement in the water column, enabling the organism to respire naturally. As a result, long-term cultivation studies suggest rotation as a preferred protocol for diverse zooplankton. Copepods were previously shown to not sink passively, but rather to sink and swim alternatingly, during which the rotation gives them more freedom of movement because the sinking component of their movement is extended along the circular path that a passively sinking particle would move. We tested running the RotoBOD™ with copepods turned 90° to the side and did not observe a significant difference between respiration using horizontal and vertical rotation (FIG. 3A). The main advantage the rotation provides to the animal incubations is a decrease in encounters with the walls of the vial and more steady mixing of the sample, which allows for very precise oxygen detection without time delays. These tests suggest that this is beneficial for the survival of the organism.

To prepare the present system for incubations, sample vials were washed in 1% hydrochloric acid to remove any contaminants without damaging the optode spot. Sample vials were filled with either seawater, 0.2 μm filtered seawater, or filtered seawater and an added particle or organism. The particle or organism was washed by transferring it to 0.2−μm filtered seawater and subsequently moved to the vial using a wide-bore glass pipette. The vial was closed and crimped, the absence of bubbles within the vial was confirmed visually. During incubations, the samples were kept at constant temperatures by placing the RotoBOD™ in a temperature-controlled room or incubator. After incubations, the absence of a bubble was confirmed again to avoid oxygen concentration changes due to outgassing or absorption.

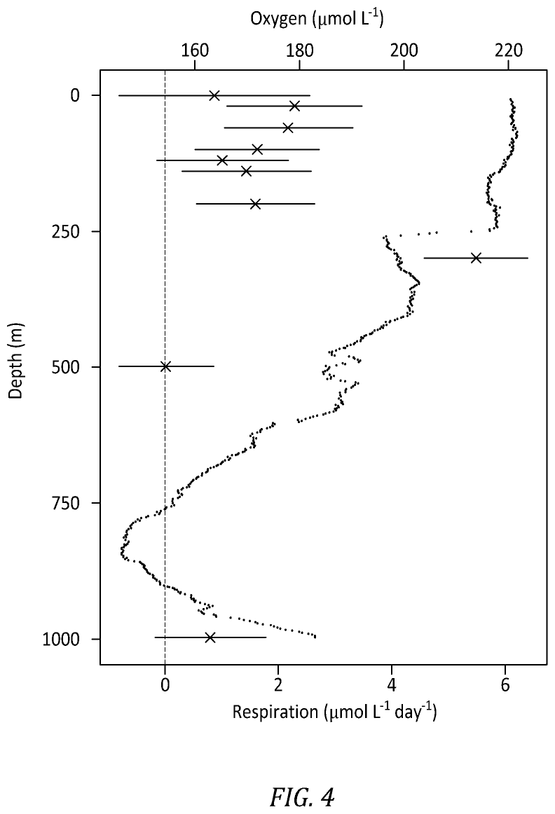

FIG. 4 depicts RotoBOD-detected respiration rates in seawater. X symbols denote oxygen respiration detected in 10 mL seawater samples at the BATS time series station, using the RotoBOD™ (for each depth, n=1, lower x-axis). Error bars represent the standard error of a Monte-Carlo-simulation histogram. Red dots represent oxygen concentrations, shown in the upper x-axis.

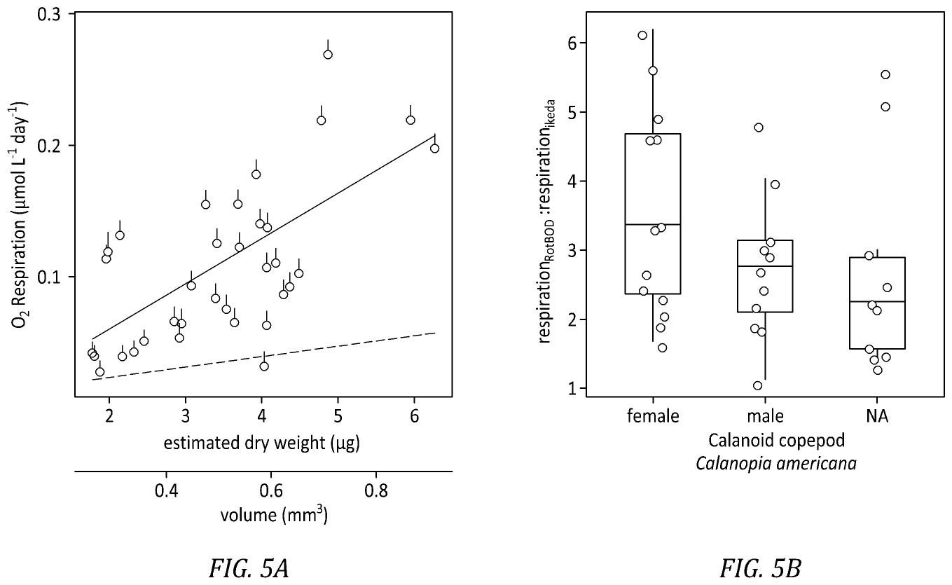

FIG. 5A depicts oxygen respiration of individual Calanopia americana copepods correlates with the animal's biovolume as estimated from microscopic images and resulting estimated dry weights. Error bars are standard errors calculated from a Monte-Carlo simulation. A quantity of 0.2−μm-filtered seawater was incubated as a blank and a resulting mean background rate of 0.004 μmol copepod−1 day−1 was subtracted from the copepod rates. The solid line represents the linear correlation between volume or estimated dry weight and oxygen respiration. The correlation is statistically significant but biovolume is likely not the sole predictor of respiration (p<0.001, R2=0.43). The dashed line represents the correlation between dry weight and respiration.

FIG. 5B shows the ratio between RotoBOD-based respiration and size-based predicted respiration in female and male individuals, and juvenile copepods or animals for which the sex could not be determined for other reasons, respectively. Based on a Student's t-test, the mean ratio is significantly higher for female copepods (ratio 3.6) than for male copepods (ratio 2.9), which suggests that the theoretical prediction underestimates respiration especially in female animals by not taking the individual copepod's reproductive physiology into account.

Oxygen concentrations were estimated based on light quenching, as is standard for optode sensors. To accurately correct measurements for temperature effects, the temperature was measured proximate to the spot using a long-wave-infrared sensor. A Monte-Carlo simulation was used to estimate respiration rates, an approach that is commonly used in time series measurements. While a linear regression yields an accurate slope, the measurement pattern of several replicate measurements in 1-second intervals repeated in 25-to 60-minute intervals results in an incorrect number of degrees of freedom, rendering statistical assessments of a linear regression inaccurate. For the Monte-Carlo simulation, a large number (>50) of linear slopes between randomly picked data points, excluding pairs of measurements with less than a minute time difference, were estimated and united in a histogram (FIGS. 2B and 2C). The mean of the resulting normal distribution represents the slope of the regression and was shown to be close to identical to a respiration rate based on a single linear regression, whereas the standard error of the normal distribution's mean is a more accurate assessment of the variability within the measurement. A custom-written R-script was used to calculate oxygen concentrations from the raw signals and correct them for on-the-spot-temperature, and to perform the Monte-Carlo-simulation. The calculations were performed using R version 3.6.1 (2019-07-05), and the packages dplyr, data.table, and broom were used within the script. Figures were plotted using the ggplot2 package and edited using Affinity Designer.

Rates in water samples were detected as a change of oxygen concentrations over time, in units of μmol L−1 day−1. Due to the vial size of 10 mL, a particle or organism's respiration of 1 μmol resulted in a concentration change of 100 μmol L−1 in the vial. Therefore, respiration per particle or organism was calculated as 1/100 of the measured concentration change and presented in units of μmol individual−1 day−1, which can further be converted to respiration per biovolume or dry weight of the particle or organism. This exemplifies how using small vials allowed the user to increase detection for this type of sample.

Particles and organisms were incubated in 0.2 μm filtered seawater from the location where the samples were collected. While the 0.2 μm filtration is supposed to remove all living cells from the sample, small contaminations in the system or due to very small cells passing the filter may lead to a background rate. Therefore, the oxygen drawdown detected was subtracted in the 0.2−μm-filtered seawater blanks from the drawdown detected in the samples. The median blank rate of 0.2−μm-filtered seawater during the incubations of the zooplankton test samples was 1.01 +/−0.81 μmol L−1 day−1 (median of all 36 0.2−μm-filtered seawater incubations during the BIOS/BATS sampling campaign). As a result, the detection limit based on the standard error during these experiments was 0.81 μmol L−1 day−1 for water samples and 0.0081 μmol individual−1 day−1 for the incubated particles or organisms. Through high replication, this detection limit was reached although it is lower than the resolution of the sensor dots (+/−1.4 umol/L), which is reflected in the variability of the replicate pulses (for example Supplementary FIG. 2) and the Monte-Carlo-simulation. Differences in absolute values between the spots do not affect the linear drawdown observed in measurements carried out repeatedly on the same position on the same spot, which renders a calibration of the spots to absolute zero unnecessary. Variability that occurred within each vial over time is reflected in the standard error of the Monte-Carlo simulation and did not vary detectably between the spots. Standard error of the rates, and background oxygen consumption detected in the 0.2−μm-filtered seawater varied, depending on the number of data points (i.e. for how long the measurement was allowed to run), sample type, absolute oxygen concentrations, variability in positioning through measurements at sea versus on land and temperature. Therefore, determining the blank rate and assessing the detection limit of each run individually through a set of background water samples is recommended.

A longer run generally allows detecting a lower rate, but most of our samples showed a detectable rate within 24 hours. Samples were generally incubated as shortly as possible to avoid bottle effects, except for stability tests with filtered water, which were run over longer periods (˜70 hours) but did not show significant changes over time. If a bottle effect occurs, it leads to a non-linear slope that can be detected in the raw oxygen data and Monte-Carlo error. Therefore, this was included an option to check all raw data in the processing script to enable manual corrections in case of any bottle effects or other disturbances.

Respiration requires an exchange of substrates and products with the surrounding water while a respiring particle sitting at the bottom of a vial can form an anoxic micro-niche, which supports an unnatural switch from oxygen respiration to anaerobic metabolisms. It was tested whether this effect can be observed operating the RotoBOD™, filled with organic particles collected at Eel Pond, Woods Hole, in its regular orientation (vertical mode; runs a and c in FIG. 3B) and rotated by 90 degrees (horizontal mode-run b in FIG. 3B). Particle morphology did not change significantly throughout the experiment, which is consistent with observations we made in other experiments of similar durations (CK unpublished data). In 10 out of 12 particles, respiration was lower in horizontal mode than in both vertical runs (FIGS. 3A-3B). In two particles, respiration increased over time (run a<b<c), which suggests growth of the associated microbial communities to be stronger than the effect of the rotation and underlines the importance of running short (<24 h) respiration measurements in active samples to ensure a linear rate and natural microbial community. Respiration in horizontal mode was below detection in many of the particles. This is likely a result of both the particle respiring less and the lack of rotation preventing the moving particle from sufficiently mixing the water within the vial. While the RotoBOD™ does not provide information on which of these two factors is most relevant, its rotating design solves both problems that would otherwise result in underestimating respiration rates.

The Bermuda Atlantic Time-series Study (BATS) provided an opportunity for a quantitative assessment of our approach. Oxygen respiration was measured in seawater at 10 water depths (FIG. 4). The resulting rates ranged between 0.02-5.50 μmol L−1 day−1 +/−1.1 μmol L−1 day−1. These results are in a similar range as in situ respiration measurements with the optode-based PHORCYS system (21) which detected 4.2 +/−0.3 μmol L−1 day−1 at 13 m water depth at a North Atlantic station in the same region as the BATS site (32° 57′ 2.4″ N 65° 44′ 58.8″ W) and a rates ranging between 1.8 +/−0.2-7.8 +/−0.4 μmol L−1 day−1 within the euphotic zone (7-29 m water depth) at diverse open-ocean locations. It should be noted that seawater rates in other studies employing optode sensors are determined over longer incubation periods than our measurements. If using the RotoBOD™ for seawater or other non-particulate samples resulting in slow oxygen drawdown, we suggest incubation periods longer than ˜24 hours.

Respiration rates were measured in diverse zooplankton and tested for linear correlations with zooplankton body size and size-derived estimated dry weight. Respiration rates of Calanopia americana copepods were elevated by a factor 4 compared with literature values. It should be noted that the animals used in our incubations were smaller than the species used by Ikeda et al., and they were not starved (i.e., kept without food for 10-24 h before incubation), as is common for zooplankton respiration measurements, both of which can explain the higher rates. Respiration per animal correlated with animal size as obtained from microscopic images (FIGS. 5A-5B). Due to its high sensitivity and replication, our method is particularly useful to study differences between individual animals that may explain variability in the size-respiration correlation. For example, a difference between female and male copepods could be observed. Respiration per body volume (FIG. 5B) and dry weight was higher in female copepods than for male copepods, and overall variability in female copepods was higher. This suggests that reproductive functions such as forming and carrying eggs in female copepods require varying levels of oxygen supply, which is consistent with previous observations. This result emphasizes the importance of carrying out measurements on single organisms and taking individual characteristics into account.

There is a long history of the use of rotation to improve culturing outcomes for small and gelatinous plankton species, which often experience high percentages of mortality when kept under laboratory conditions. A substantial part of early work with copepod culturing was done with the plankton wheel, which shares some similar design elements to the RotoBOD™, while the success of long-term jellyfish cultivation and other gelatinous planktonic species, has proven to be contingent on the use of rotational flows. Long-term cultivation intrinsically requires an organism to respire above its basal metabolic rate. Similar to those approaches, the RotoBOD™ provides a low-stress method of keeping a specimen in constant motion while carrying out respiration measurements.

It was tested whether the direction of rotation affects the outcome of respiration measurements on the benthic copepod Stygiopontius hispidulus. A t-test revealed that there was no statistically significant difference between horizontal and vertical rotation mode (p-value 0.95, n=20). While the median respiration rates of 0.028 and 0.024 μmol O2 animal−1 day−1 for horizontal and vertical rotation were variable due to their low range and short incubation time, they were significantly different from the median blank rate (median −0.017 μmol O2 animal−1 day−1, p=0.01) that was subtracted from all sample rates. Therefore, our observation suggests that rotation is an approach that does not affect small zooplankton negatively and can therefore be used for respiration measurements in vials that are automatically mixed through the animal's sinking and provide space for it to move naturally. However, it should be noted that zooplankton are taxonomically, morphologically, and functionally highly diverse, which comes with different densities, behaviors, and swimming patterns. This variability likely influences their response to ex situ respiration rate measurements, with some animals potentially being more disturbed by encountering the bottom of a stationary vial, while others may be more stressed by rotational movement. Further exploration of the variability in metazoan metabolic rates using multiple respiration setups may reveal important patterns in taxonomic response and should be the topic for further research.

To provide further biological and chemical oceanographic details, it is noted that C. americana is a prevalent copepod found in the coastal Western Atlantic, ranging from approximately 35° N to 30° S. Primarily associated with shallow environments, C. americana displays a distinctive twilight Diel Vertical Migration pattern. This migration involves ascending to the surface at sunset, migrating near the bottom around midnight, followed by a second ascent to the surface and subsequent descent to the bottom at sunrise. During the day, C. americana buries itself in the sediment as a protective measure against predators in the water column. This calanoid is smaller than most metazoan zooplankton used in individual respiration experiments (˜1 mm), and thus serves as a good model for the highly abundant but poorly characterized smaller planktonic metazoans. Small zooplankton introduce high uncertainties to current earth system models because patterns in their complex ecology and contribution to carbon remineralization are unclear, which emphasizes the importance of studying them under controlled near-natural conditions. Stygiopontius hispidulus are a small (<1 mm) species of deep-sea copepods known to colonize new hydrothermal vent habitats. They were observed to move fast, which enables them to live in unstable conditions, and which makes them a good model to test for the effect of rotation on animal movement.

Detritus particles form from decaying biological material near the sea surface and play a central role in carbon flux and sequestration in the ocean. As they form from diverse source materials and may further be ballasted through aeolian dust input, each particle is unique in its composition and morphology. Further physical aggregation and disaggregation while the particle sinks and becomes a source of nutrition for diverse organisms shape its composition, morphology, and physico-chemical properties at depth. Characterizing particles individually is essential to link their properties to particle-associated remineralization rates, or to predict carbon flux and remineralization based on underwater camera technology more accurately in future earth system models. The particles we use as test samples originate from a shallow marine bay and their physical properties are representative of fresh sinking biomass and enable us to observe processes that are likely to take place on diverse particulate samples due to sinking.

Seawater samples were collected at the BATS (Bermuda Atlantic Time-series Study) site (32° 10′ N, 64° 30′ W). Zooplankton were collected during two sampling campaigns. First, animals were collected by manually towing a 150 μm net at the dock of Bermuda Institute of Ocean Science (BIOS, 32.3709 °N, −64.6962° W) for five days shortly after the onset of darkness. Copepods, crab larvae, jellyfish and juvenile shrimps were isolated and transferred into 0.2−μm filtered seawater and incubated in the RotoBOD™ at 20° C. in the darkness for up to 36 hours. After incubation the animal's survival was confirmed based on visual movement under the microscope. A scaled microscopic image of each metazoan was taken (Leica M205 C Stereomicroscope with an Infinity3 Lumenera camera). Zooplankton bio-volumes were determined from the images, using ImageJ software to determine the length and width of each organism, and assuming an ellipsoid body shape to calculate volume. Dry weight was estimated from biovolume using the equation for copepods from Maas et al. 2021. The correlation of dry weight with respiration described by Ikeda et al. 2014 using larger copepods was applied to estimate theoretical respiration in our C. americana and compare it with detected respiration rates.

Second, small benthic copepods of the species Stygiopontius hispidulus were collected at an active hydrothermal vent site on the East Pacific Rise (9° 50′ N, 104° 17′ W, 2514 m water depth) using a suction sampler. They were transferred into 0.2−μm filtered seawater from the same site and incubated in the RotoBOD™ at 5° C. in the darkness under regular (vertical) and horizontal rotation for 10-16 hours each.

Organic detritus particles were collected at Eel Pond, Woods Hole (41° 31′36″ N 70° 39′47″ W) during high tide, using a bucket with a lid. Sinking particles accumulated at the bottom of the bucket and were picked out individually using a wide-bore pipette.

Alternative constructions and methods include varying the RotoBOD's bottle sizes, which is easy to implement due to the 3D-printed design. Besides keeping individual samples afloat, our instrument can enable homogenous mixtures of samples such as microbial cultures to be kept in gentle motion, with the added benefit of constant oxygen monitoring. Due to the non-invasive detection on individual samples, the RotoBOD™ system allows for studies that link respiration on individual samples with their unique characteristics, and their abundance and role in the environment. Visual properties of incubated marine particles and organisms can be used to extrapolate oxygen consumption rates to the water column using camera systems. Mechanistic links between respiration and sample properties can be derived by combining this new technology with measurements on the same samples after incubations, such as microscopy, genetic analyses, elemental analyses, and dry weights.

Different uses (i.e., applications) of the present RotoBOD™ system include measuring various types of respiration, micro-aerobic respiration, anoxic incubations, and photosynthesis measurements. Various sizes of particulate organic samples obtained from different aquatic environments can be incubated and measured while simulating sinking experienced naturally in those environments. Regarding planktonic organisms, it has been shown that zooplankton prefer rotating incubators as it allows them to move in the water at near-natural conditions. Further, the rotation ensures homogeneous mixing of the surrounding water, which is important when estimating oxygen drawdown based on concentration measurements. Regarding microplastic degradation, the microplastic particles must remain suspended throughout the incubation to be accessible to bacterial degradation.

Additionally, heterogeneous samples benefit from gentle mixing that can be provided by the RotoBOD™ system. An example is a microbial cultivation experiment in which we test a microbial strain's ability to metabolize carotenoids. Carotenoids are hydrophobic. To expose the microbes to carotenoids isolated from other organic compounds, we attached the carotenoids to glass beads, which are kept in motion and thereby accessible to the bacteria through the rotational movement of the RotoBOD™ system. Another example with similar properties would be a sediment slurry sample.

For micro-aerobic respiration, RotoBOD™ systems operating with trace oxygen sensor spots such as illustrated in FIG. 1E enable incubations at low-oxygen conditions.

Diverse experiments, such as stable isotope tracer amended nitrogen cycling measurements, happen at anoxic conditions. Often, it is difficult to ensure that no oxygen contamination occurred during sample transfers into the vial. The RotoBOD™ system enables oxygen monitoring in large numbers of samples, and the bottles are suitable for subsampling through the rubber lid.

Regarding photosynthesis measurements, oxygen is produced during photosynthesis, which we detected successfully when incubating small kelp individuals with light. When the light was switched off, we were able to detect the same individual's respiration, which enables detailed ecological studies. If the kelp were not kept in motion by the RotoBOD™ system, oxygen would not be distributed homogeneously in the vial, introducing a source of variability and error, especially as a lag would be introduced during switches between light/dark settings.

The term “portion” as utilized herein refers to a section or region of a component, without necessarily indicating any physical difference between two or more portions apart from location such as “upper portion” and “lower portion”.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they may be merely conceptual in nature.

It is to be understood that the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions. Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on, or executable by, a programmable computer including any combination of any number of the following: a processor, a storage medium readable and/or writable by the processor (including, for example, volatile and non-volatile memory and/or storage elements), an input device, and an output device. A processor is also referred to herein as a controller, a microcontroller, or a processing resource. The input device and/or the output device form a user interface in some embodiments. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention automatically provide sequential x-y positioning of a sensor with selected sample vials at timed intervals, automatically obtain sensor measurements of those vials, and automatically update data in an electronic memory representing such measurements. Such features can only be performed by computers and other machines and cannot be performed manually or mentally by humans.

Any claims herein which affirmatively require a computer, a processor, a controller, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a controller, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language. Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays).

A computer can generally also receive (read) programs and data from, and write (store) programs and data to, a non-transitory computer-readable storage medium such as an internal disk (not shown) or a removable disk or flash memory. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium or other type of user interface. Any data disclosed herein may be implemented, for example, in one or more data structures tangibly stored on a non-transitory computer-readable medium. Embodiments of the invention may store such data in such data structure(s) and read such data from such data structure(s).

It is the intention, therefore, to be limited only as indicated by the scope of the claims appended hereto. Other embodiments will occur to those skilled in the art after reviewing the present disclosure and are within the following claims.

What is claimed is:

1. A system to incubate and monitor liquid samples over a selected time period, comprising:
at least one rotatable cassette having an axis of cassette rotation and configured to hold a plurality of sample vials, the vials being held radially outwardly from the axis of cassette rotation along a plurality of spaced radii relative to the axis of cassette rotation;
each vial having a sensing location, and each vial having a longitudinal axis alignable by the cassette with one of the spaced radii to position its sensing location proximal to the axis of cassette rotation;
a sensor configured to measure at least one parameter and having a sensor head;
a first motor configured to rotate the cassette about its axis of cassette rotation; and
a controller configured to actuate the motor to periodically align the sensing location of each vial with the sensor head to measure the at least one parameter of that vial.

2. The system of claim 1 wherein each vial has at least one optode spot at its sensing location.

3. The system of claim 1 including at least two cassettes, and the sensor is mounted on a sensor track to enable shuttling between the cassettes.

4. The system of claim 3 wherein the sensor track includes at least one guide rail and an advancement mechanism.

5. The system of claim 4 wherein the advancement mechanism includes a second motor configured to shuttle the sensor between the cassettes.

6. The system of claim 3 wherein the cassettes are rotated together by the first motor in fixed alignment with each other.

7. The system of claim 1 wherein the sensor measures at least oxygen concentration of the liquid sample within each vial.

8. The system of claim 7 wherein the temperature of the liquid sample within each vial is determined when the oxygen concentration is measured.

9. The system of claim 1 wherein at least one cassette is configured with a releasable vial locking mechanism to enable individual removal and replacement of at least one of the vials from the cassette assembly.

10. A method for incubating and monitoring liquid samples over a selected time period, comprising:
selecting at least one rotatable cassette having an axis of cassette rotation and configured to hold a number of sample vials, the vials being held radially outwardly from the axis of cassette rotation along a plurality of spaced radii relative to the axis of cassette rotation, and each vial having a sensing location and having a longitudinal axis alignable by the cassette with one of the spaced radii to position its sensing location proximal to the axis of cassette rotation;
placing a liquid sample in each of a plurality of sample vials;
rotating the cassette at a selected rotation speed at selected intervals to achieve a selected sampling period for each vial; and
measuring at least one parameter within each vial utilizing a sensor head by periodically aligning the sensing location of each vial with the sensor head to measure the at least one parameter of that vial.

11. The method of claim 10 wherein the rotation speed is selected to be sufficiently high to prevent planktonic items in the liquid sample within each vial from accumulating on any surface of that vial.
